(19) United States
(12) Patent Application Publication
(10) Pub. No.: US 2007/0032748 A1
(43) Pub. Date: Feb. 8, 2007

(54) System for detecting and analyzing body motion

(75) Inventors: Russell McNeil, Surrey (CA); William Hue, Port Coquitlam (CA); Pete Rizun, Calgary (CA); Alex Jiang, Surrey (CA); Michael Lee, Vancouver (CA)

Correspondence Address: VERMETTE & CO., SUITE 320 - 1177 WEST HASTINGS STREET, VANCOUVER, BC, V6E2K3, CA

(73) Assignee: 608442 BC Ltd.

(21) Appl. No.: 11/190,945

(22) Filed: Jul. 28, 2005

(51) Int. Cl. A61B 5/11

(52) U.S. Cl. 600/595; 73/865.4; 73/509; 73/510

(57) ABSTRACT

A portable sensor system that uses acceleration-insensitive, three-dimensional angle sensors located at various points on the patient's body, and collects data on the frequency and nature of the movements over extended periods of time.

### FIELD OF INVENTION

The present invention relates to sensor systems for performing functional assessments of biomechanics.

### BACKGROUND OF THE INVENTION

A capacity assessment (also referred to as a functional capacity evaluation of biomechanics (FAB) or functional capacity evaluation (FCE)) is a test of a person's ability to perform functional activities in order to establish strengths and weaknesses in executing daily activity. Functional activities are defined as meaningful tasks of daily living, including personal care, leisure tasks, and productive occupation. Productive occupation does not strictly mean paid employment. Productive occupation can be any daily activity that occupies time including housework and yard work.

Presently there is no practical system available for sensing biomechanics or body positions during testing, rehabilitation, and daily tasks. Traditionally a therapist, physician or chiropractor observes a patient and assesses biomechanics and body positions through best guess techniques, which is an imprecise and time-consuming process. Further, once information is gathered there is usually a slow and cumbersome process of incorporating data into formal reports.

In order to obtain the type of data needed to conduct a capacity assessment, body movements must be measured simultaneously in 3 planes, namely, frontal, sagittal and transverse (e.g. lower back movements) (see FIG. 1). Body motion tracking is often limited to laboratories equipped with camera-based tracking systems (see, for example, systems developed by Human Performance Labs, and area-limited performer-trackers, such as those of Ascension MotionStar). Because of the complex optical environment required, measurements outside of the laboratory are notoriously difficult. Portable solid-state angle sensors such as those employing accelerometers—although accurate for static measurements—are not suitable for body motion tracking. They are highly sensitive to the accelerations associated with normal human movement. There are inherent problems with trying to measure relative position and absolute velocity with these types of sensors without correcting for inertial effects (in the case of accelerometers) and integration offset (in the case of gyroscopes). Accordingly, there is a need in the art for a comprehensive tool to augment capacity and disability testing by sensing body positions and movements over extended periods of time.

A portable device that could be attached to a part of the body and accurately measure its orientation could have numerous applications.

### SUMMARY OF THE INVENTION

The present invention is a portable sensor system that allows a Functional Capacity Evaluation to be efficiently and accurately performed on a patient. The invention is herein referred to as the FAB System. The present invention uses 3D sensors located at various predetermined points on the patient's body, and collects data on the frequency and nature of the movements over extended periods of time (e.g. from 8 up to 35 hours).

The present invention comprises, in part, a novel acceleration-insensitive, three-dimensional angle sensor employing magnetometers, accelerometers and gyroscopes. The angle sensor, in conjunction with a novel computation, provides for accurate measurement of body position. The gyroscope angular velocity measurements—unaffected by acceleration—are used to continuously rotate a matrix that represents the orientation of the sensor. The magnetometer and accelerometer measurements are used to construct a second matrix that also estimates the sensor's orientation—unsusceptible to the drift introduced by integrating the gyroscope signals. The first matrix is “pulled” slightly towards the second matrix each step of the computation, thereby eliminating drift. The result is a high-bandwidth orientation sensor that is insensitive to acceleration.

The 3D sensor performs acceleration measurement along 3 axes, inertial (gyroscopic) measurement along 3 axes, and magnetic measurement along 3 axes. Several different embodiments of the 3D sensor are contemplated. For example, since some commercially available accelerometers and magnetometer chips have 2 axes per chip, one economical embodiment of the 3D sensor is made up of 2 accelerometers, 3 gyroscopes, and 2 magnetometers (i.e. only 3 of 4 available accelerometer and magnetometer axes would be used). Alternatively, the 3D sensor could be built using 3 accelerometers, 3 gyroscopes and 3 magnetometers, each having one axis. In each of these embodiments the 3D sensor is capable of performing acceleration, gyroscopic and magnetic measurements along 3 axes.

The primary application of the invention is in the diagnosis and rehabilitation of orthopaedic injuries such as fractures and connective tissue injuries such as back injuries or shoulder injuries. However, the invention can also be used for neurological injuries such as stroke or nerve injuries. The invention generally has application in medical conditions where there is restricted movement of the arms (shoulders), spine, legs, or feet.

The 3D sensors of the present invention have numerous potential medical and non-medical applications.

It is envisioned that the invention will be used primarily in rehabilitation clinics, work places, and at home. For example, if a housewife were in a motor vehicle accident and had a whiplash injury with back pain, the sensors could be used to monitor movement while at home. However, the system can generally be used as necessary in any setting or environment.

In a preferred embodiment there are 4 sets of paired sensors, one pair for the feet, one for the legs, one for the spine, and one for the arms. The sensors on the legs, lumbar spine, and the arms are 3D sensors. The foot sensors are pressure sensors. The invention can be used with either a partial or a full complement of sensors so that end users can purchase, use and/or configure the invention as needed.

More sensors can be added to obtain more detail about the movements of various parts of the body. For example, sensors could be placed on the ankles, on the wrists, between the shoulder blades, on the back of the neck and/or head, etc. The number and placement of the sensors is a function of the type and amount of data needed.

The data provided by the 3-D sensors is processed according to algorithms that calculate the path-independent angles between any two 3-D sensors.

### BRIEF DESCRIPTION OF THE DRAWINGS

Further features and advantages will be apparent from the following Detailed Description of the Invention, given by way of example, of a preferred embodiment taken in conjunction with the accompanying drawings, wherein:

FIG. 1 shows the frontal, sagittal and transverse planes and axes;

FIG. 2 is a diagram of a preferred embodiment of the invention as worn by a patient;

FIG. 3 shows layout of a preferred embodiment of the 3D sensor;

FIG. 4 shows a 3D sensor attached to a patient's leg;

FIG. 5 shows angles sensed by an accelerometer-based sensor placed above a patient's knee, 40 cm from the point of rotation;

FIG. 6 shows the angle sensed by a gyroscope-based sensor for a patient moving his leg between 0 degrees and 90 degrees at 30 repetitions per minute;

FIG. 7 shows a plot of estimated and actual angles of a patient's leg moving between 0 and 90 degrees;

FIG. 8 shows a plot of patient's leg moving between 0 and 90 degrees;

FIG. 9 shows a general architecture of the FAB Software;

FIG. 10 illustrates definitions of angles of the patient's arms in the frontal and sagittal planes;

FIG. 11 illustrates definitions of angles of the patient's arms in the transverse plane;

FIG. 12 illustrates rotation of a vector r about n by the finite angle Φ; and

FIG. 13 illustrates the spherical angles of a patient's arm.

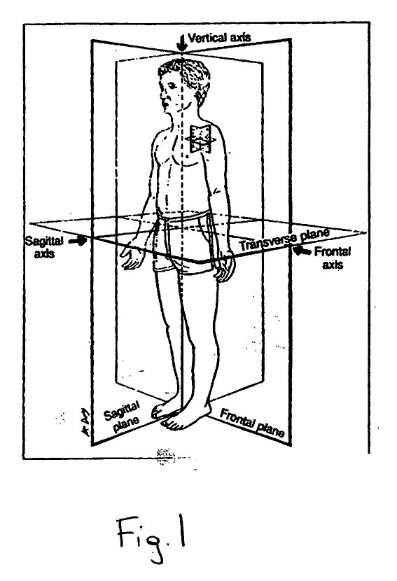

### DETAILED DESCRIPTION OF THE INVENTION

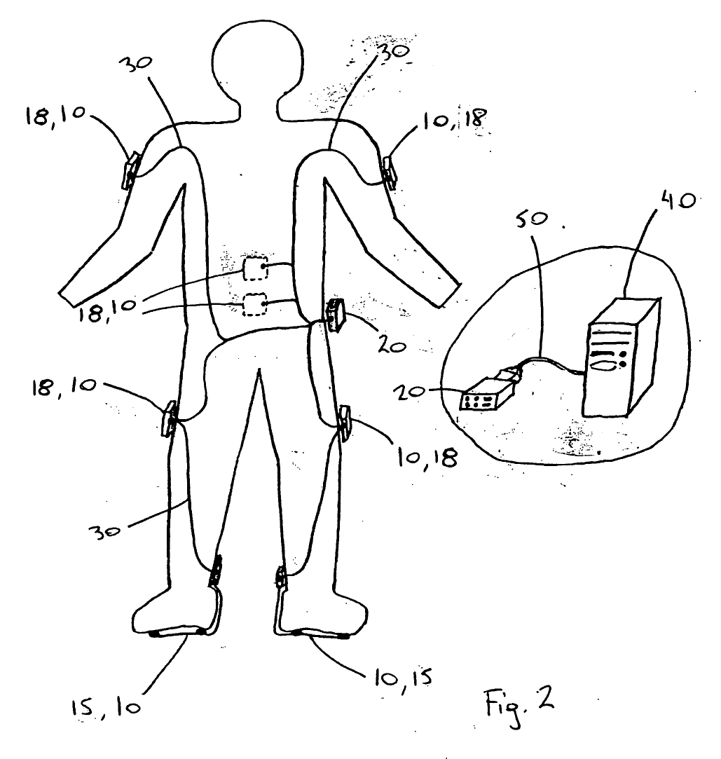

The system consists of a number of sensor units 10 and a belt-clip unit 20, as shown in FIG. 2. All of the sensor units 10 are connected to the belt-clip unit either wirelessly or by cables 30. In a preferred embodiment EIA RS-485 is used as the physical communication layer between the units. There are two types of sensor units 10, foot sensors 15 and 3D sensors 18.

A patient will wear the system for an assessment period, which is long enough for the system to gather the required amount of data. Although the system may be worn by a patient for periods of up to 24 hours or more, (depending on available battery power and memory) it is contemplated that assessment periods will generally be up to about 8 hours long. Data can be gathered over several assessment periods.

Data collected during an assessment period is stored in the Belt Clip 20 and can be subsequently transferred to a computer 40. In one embodiment, data is transferred by connecting an EIA RS-232 serial cable 50 between the Belt Clip 20 and the computer 40. Alternatively, the data can be stored on a memory card, which can be used to transfer the data to a computer.

Sensors

In the embodiment of FIG. 2, all sensor units 10 are connected directly to the Belt Clip 20 via combined RS-485/power cables 30. Each sensor unit 10 has an on-board microprocessor, and is powered and controlled by the Belt Clip 20. Data from the sensor units 10 is sent to the Belt Clip 20 over the RS-485 signals in real time.

All of the sensor units 10 are able to sense and record duration and number of repetitions of movements. In order to detect rapid movements, sensor units 10 must be able to provide readings or measurements more than once a second. In the preferred embodiment the sensor units 10 provide readings 25 times per second to the Belt Clip 20.

In a preferred embodiment the invention is modular in the sense that it is made up of a number of interchangeable sensor units 10 (although the foot sensors 15 are obviously not interchangeable with the 3D sensors 18 because they perform different functions). End users can configure the system according to their needs or for a particular application by adding or removing sensor units 10.

In an alternate embodiment the system is wireless and the individual sensor units 10 are connected to the Belt Clip 20 by 900 MHz or 2.4 GHz transceivers. The transceivers of the wireless embodiment replace the cables 30 used in the embodiment of FIG. 2. Additional minor modifications may be necessary in the wireless embodiment, for example to the communications protocols to accommodate for the imperfect nature of wireless communications. In addition, each wireless sensor unit will have its own battery and power management hardware and/or software.

Foot Sensors

In the preferred embodiment sensors are placed under each foot to measure weight and pressure. The foot sensors 15 are able to sense pressure in both the heel and ball of the foot. The foot sensors 15 precisely measure applied pressure. Depending on the way the patient's weight is distributed on his or her feet, the pressure reading may or may not directly translate to an accurate weight reading, however, the pressure readings will be consistent.

The foot sensors 15 are flat. Each foot sensor 15 will consist of an ankle “bracelet” (attached to the ankle with a Velcro® strap) containing the electronics and an attached flexible “tongue”. The tongue contains the pressure sensors to be placed under the sole of the foot, normally inside a shoe.

Leg, Arm and Back Sensors

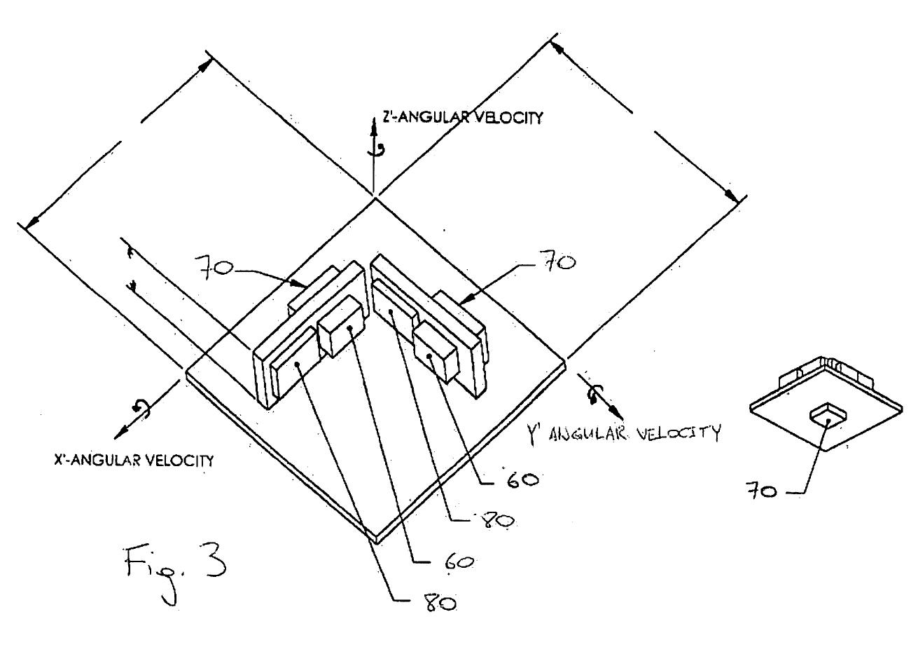

Each “3D” sensor 18 (that is, a sensor that is not a foot sensor 15) is a device that is designed to sense yaw, pitch and roll relative to the earth, with no physical attachment to the ground. Each 3D sensor 18 comprises components that allow it to measure each of rotational velocity (gyroscopes), gravitational pull (accelerometers), and the earth's magnetic field (compasses), in three orthogonal directions x, y and z (see FIG. 3). The sensor components are mounted such that all three are measured along the same x, y and z axes. Commercially available accelerometers and compasses generally have two axes each, therefore, two accelerometers 60 are sufficient to cover the 3 axes in the embodiment of FIG. 2. The embodiment of FIG. 2 has three gyroscopes 70 and two compass chips 80.

Theoretically, for the 3D sensors 18 all that would be needed would be gyroscopes 70 to measure movement, since position is the time-integral of velocity (and therefore can be calculated from velocity data). However, real gyroscopes aren't perfect and their readings always contain drift and offset from zero. Additionally, the Earth's Coriolis effect will give a small, but nevertheless non-zero reading even if the sensor is stationary.

Depending on the specific components used to construct a sensor 18 according to the present invention, sensitivities of the sensors 18 may vary (e.g. certain motions or signals may have to be filtered out in the hardware and/or software, and/or delays may have to be introduced).

Preferably, all components are placed as close to one another as possible to reduce Ω²r acceleration. If possible the lines through the centers of the gyroscopes 70 and accelerometers 60 are perpendicular to their surfaces and intersect at a common point (see FIG. 3).

Leg Sensors

A leg sensor is placed on the right and left thighs proximal to the knee (see FIGS. 2 and 4). The leg sensors accurately measure angles relative to gravity in the sagittal plane. The leg sensors are attached to the thighs with Velcro® straps or other suitable means.

Arm Sensors

In the preferred embodiment, (FIG. 2) arm sensors are attached to the arms just above the elbow on the lateral side (outside). The arm sensors accurately measure angles relative to gravity and magnetic North in the sagittal, frontal and transverse planes. The arm sensors are attached to the arms with Velcro® straps or other suitable means.

Back Sensors

The back sensors consist of two or more units which are capable of measuring angles in the sagittal, frontal and transverse planes. Measurement in the transverse plane poses a challenge because gravity cannot always be used as a reference. In fact, any movement in a plane perpendicular to gravity poses the same challenge.

The back sensors must be able to measure and record the range of motion of the following movements:
i. flexion (forward bending);
ii. extension (backward bending);
iii. lateral flexion (sideways bending); and
iv. rotation (twisting to the right or left).

In the preferred embodiment one of the back sensors is placed at the vertebral level of Thoracic 12-Lumbar 1 (T12-L1) and the other back sensor is placed at the vertebral level Lumbar 5-Sacral 1 (L5-S1).

The back sensors measure range of motion which is defined as the difference in the angle between the lower sensor and the upper sensor. For example, if in flexion the lower sensor moves 5° and the upper sensor moves 60°, then the flexion of the back is 55° (i.e. the pelvis tilted 5° and the back flexed 55° to result in a 60° change in position of the upper sensor).

The back sensors are able to detect combined movements (e.g. when a patient's back is simultaneously extended backwards, (i.e. in the frontal plane) twisted, and flexed laterally).

One measurement plane of the back sensors will almost always be perpendicular to gravity when the patient is in the in the normal (erect) body position. Therefore, non-gravitational sensing devices (e.g. gyroscopes) must be employed along with advanced mathematical techniques to achieve practical measurements in the transverse plane.

The sensors are attached to the lower back using a flexible adhesive, (such as a suitable adhesive tape) keeping in mind that it is desirable for the sensors to remain firmly attached during moderate perspiration. The present invention endeavours to minimize interference with normal patient activities and range of motion.

Belt Clip

The Belt Clip 20 is a compact unit containing batteries, a microprocessor, data memory and a serial interface. In the embodiment of FIG. 2 power is fed to all the sensors 10 from the Belt Clip 20. The Belt Clip 20 is able to power itself and all the sensors 10 continuously for 10 hours without having to change the batteries (in a wireless embodiment each of the sensors would have its own battery).

The microprocessor serves to collect data, in real-time, from all the sensors 10 and to store the raw information in its data memory. The contents of the Belt Clip's data memory can be transferred to a computer 40 via the serial interface, 50 which in the embodiment of FIG. 2 is a RS-232 interface (alternatively, a memory card such as a Secure Digital card may be used). The Belt Clip 20 has indicator lamps and/or buttons to indicate its operating status and facilitate calibration.

Adding up all the data from the sensors, 10 a large amount of information streams into data memory in the Belt Clip 20 every second. Although it would be technically possible to perform robotics (e.g. yaw, pitch, roll) and other (e.g. angles between sensors) calculations in the sensors' and/or Belt Clip's microprocessors, the sheer volume of data, coupled with the complexity of the computations, means that the necessary components would significantly drive up the cost of the invention. Accordingly, in the preferred embodiment, the data is stored in the data memory in the Belt Clip 20 for post-analysis. Real-time positional data would also be possible with, for example, a fast RF link to a personal computer where the data could be analyzed in real time.

An audible alarm is incorporated to indicate status changes and fault conditions (e.g. alerting the user of a low-battery or detached-wire condition).

The Belt Clip 20 is configured for a sensor “suite” by connecting all the sensors 10 in a desired configuration and executing a “configure” command via a specific button sequence. Thus, the patient cannot inadvertently leave out a sensor 10 when collecting data for the assessor.

A calibration is performed to establish a baseline for the sensor readings. This step can be performed by standing in a pre-defined position, and giving a “calibrate” command via the buttons. The calibration must be performed prior to collecting data, but may be performed additionally during data collection as desired.

The Belt Clip 20 will start collecting data from the sensors 10 once it is switched on, a calibration is performed, and a “start” command is given via the buttons. Data collection will stop if a “stop” command is given. Preferably, the stop command requires either a verification step or other mechanism to prevent accidental deactivation.

The Belt Clip 20 contains enough data memory to store, for example, 40 continuous hours of sensor information. These 40 hours may be split into multiple “sessions”, separated by “stop” and “start” commands. The data analysis software is able to distinguish one session from another.

Once data has been collected, the data is transferred to a computer. Once the data has been transferred, the data memory may be cleared with a file-operation command on the computer.

In the preferred embodiment the data memory retains its contents even if the system is shut off and/or the batteries removed.

Interconnections

In the embodiment of FIG. 2, multi-conductor RS-485/power cables 30 are used to interconnect the sensors 10 and Belt Clip 20. The cables 30 may be simply run under clothing, or secured to the body with Velcro® straps and/or adhesive tape.

In the preferred embodiment the cables 30 terminate in connectors, to allow modularity and flexibility in the configuration of the cable “network”. For example, cables 30 may be star-connected to the Belt Clip 20 and/or daisy-chained as desired.

Firmware

Firmware for the Belt Clip 20 and each sensor enables data collection, system communication and system error-checking. The firmware is the “brains” of the system and enables the sensors 10 to send data to the Belt Clip 20 by creating a two-way communications protocol over the RS-485 cables 30.

The Belt Clip firmware may have a data-transfer protocol for the RS-232 interface or a filesystem for the memory card. The firmware also performs checks on the data and hardware to ensure that faults are clearly identified. These checks help avoid collecting useless information in the event there is a system fault.

Software

The computer software collects the data stored in the Belt Clip 20, and performs mathematically-complex processing in order to interpret the data. The data will be stored on the computer hard disk, and displayed in a meaningful manner to the assessor (e.g. therapist).

The computer software interprets the measured data as physical body positions (i.e. standing, sitting, walking, etc.) and displays both the interpreted and raw data in tabular and graphical formats. The software can determine the number of repetitions performed for a variety of defined movements, the range of motion of the defined movements, the speed of movement, average or mean time spent in defined positions and/or performing defined movements, total time spent in defined positions and/or performing defined movements, maximum and minimum amounts of time spent in defined positions and/or performing defined movements, etc.

The data may additionally be stored in a relational database (RDB). This allows data to be organised, indexed and searched in a flexible manner. Additionally, third-party software can be used to generate reports from the data.

The following is a specification of a prototypical embodiment of a software program (referred to throughout as the FAB Software) for implementation of the invention. The FAB Software program is used to display information collected from the FAB system sensors. The FAB Software interacts with the Belt Clip of the FAB system and obtains recorded sensor readings, interprets the readings and displays the information in a meaningful manner. The following description of the FAB Software is intended only as a description of an illustrative embodiment. The following description is not intended to be construed in a limiting sense. Various modifications of the illustrative embodiment, as well as other embodiments of the invention, will be apparent to persons skilled in the art upon reference to this description.

FAB Software Architecture

The FAB Software will be a program, running on a personal computer (PC), that facilitates the interface between the end user and the collected data from the Belt Clip and sensors. The FAB Software is written in an object-oriented manner and is event driven. Events are generated by the user's interaction with the software's graphical user interface (GUI). Object-oriented programming helps to modularize the code making it clean and easy to understand which eases software enhancements in the future.

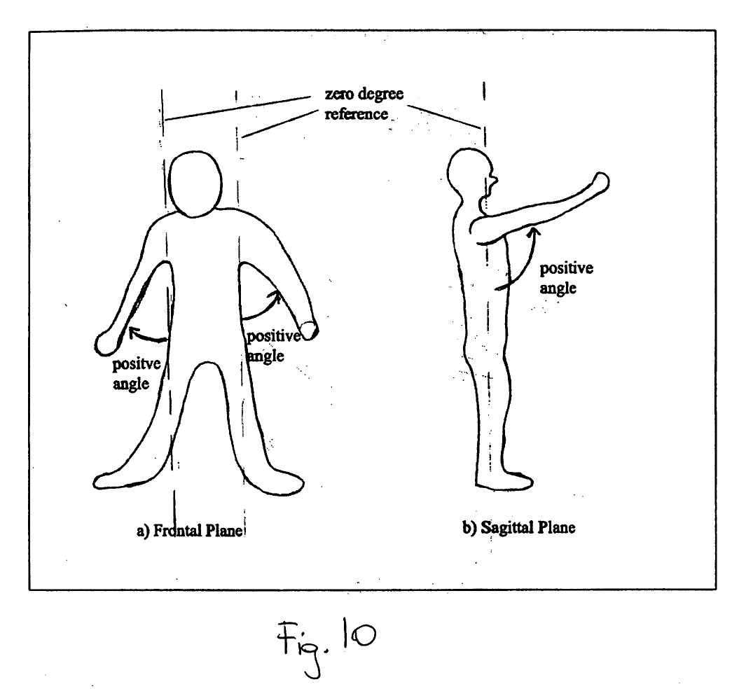

A general architecture of the FAB Software is shown in FIG. 9. The major components of the FAB Software are described in the following subsections.

Graphical User Interface (GUI)

The GUI portion of the FAB Software is built into a familiar Windows™ based framework with all the necessary displays and controls.

The GUI is also the event driver for the FAB Software. In an event driven program, all program executions occur as a result of some external event. In our case, the user is the only source of events and causes events only through interaction with the GUI portion of the FAB Software.

Belt Clip Interface

The FAB Software includes an interface to the FAB system's Belt Clip to be able to read the raw data collected by the Belt Clip. Herein, raw data is referred to as collected data in the form that it is received from the Belt Clip.

For the purposes of setting the Belt Clip's date and time clock, the FAB Software communicates with the Belt Clip via a Serial Port Interface that provides services for accessing the PC's serial port. The FAB Software's GUI provides the user a means of choosing either COM1 or COM2.

Raw data stored by the Belt Clip is organised into a number of sessions, each session being stored as a file. After all the raw data from a particular session has been obtained the Belt Clip Interface triggers the Data Interpretation Module to interpret the raw data.

Interpreting Data

After the Belt Clip has finished downloading a session it triggers the Data Interpretation module to interpret the raw data into physical body positions.

There are two stages involved in interpreting the data, a transformation stage and an interpretation stage.

Data Transformation

The raw data obtained from the Belt Clip will be raw sensor readings. These data readings must be transformed through complex differential equations to obtain the actual angle and pressure readings.

Data Interpretation

The resulting angle and pressure data obtained through the data transformation stage is interpreted to obtain physical body positions.

Data Storage

A relational database can be used to store both the raw data and the interpreted body positions. Each session can be contained in a single database.

Exporting Data

The FAB Software is able to export data tables as comma-separated variable (CSV) files, which are compatible with many other applications including Microsoft Excel®. Additionally, it may be possible for third-party software to read data directly from the database.

Example Definitions of Angles and Positions

As used herein, the terms flexion and extension refer to movement of a joint in the sagittal plane. Abduction and adduction refer to movement of a joint in the frontal plane.

The angles of the legs are only in the sagittal plane and are relative to gravity. When the leg is in the neutral position parallel to gravity the angle is defined to be 0°. The leg is defined to have a positive angle when it is angled forward in front of the body. The leg is defined to have a negative angle when it is angled backwards behind the body.

The angle of the back is defined to be negative in the sagittal plane when the back is bent forward and positive when the back is bent backwards.

The angle of the back is defined to be positive in the frontal plane when the back is bent to the right hand side and negative when the back is bent to the left hand side.

The angle of the back is defined to be positive in the transverse plane when the back is twisted to right and negative when the back is twisted to the left.

The arms in the frontal plane are defined to be 0° when the arms are in their neutral position parallel to the torso of the body. Positive angles are defined when the arms are raised to the sides as shown in FIG. 10(a). When the arms are crossed in front of the body the angles are defined to be negative in the frontal plane.

The arms in the sagittal plane are defined to be 0° when the arms are in their neutral position parallel to the torso of the body. Positive angles are defined when the arms are raised forward in front of the body as shown in FIG. 10(b). When the arms are raised backwards behind the body the angles are defined to be negative in the sagittal plane.

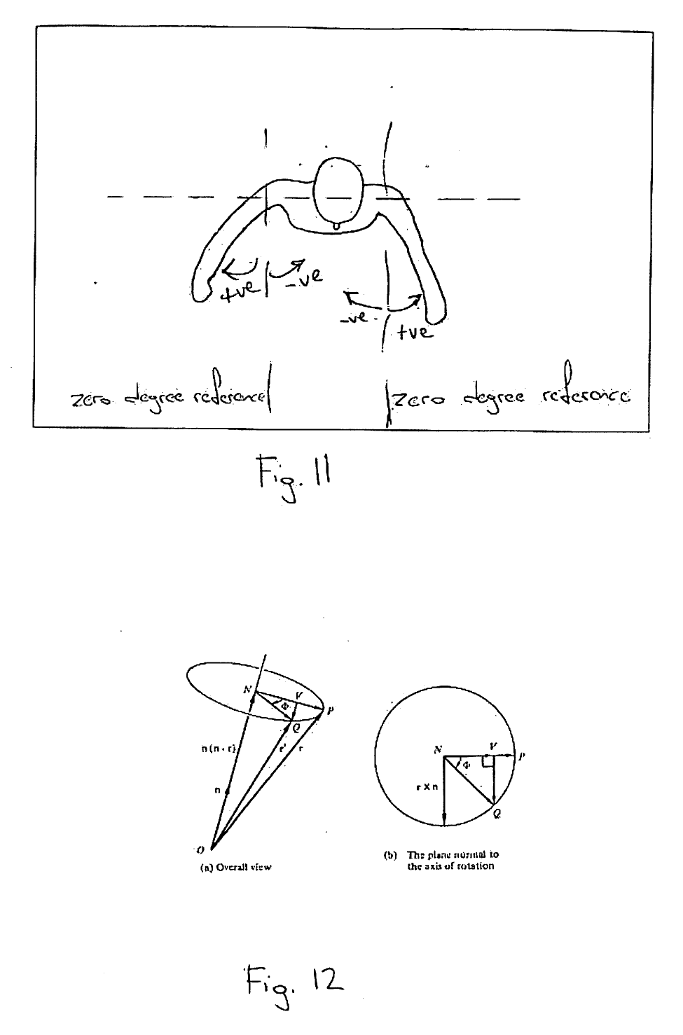

The angles of the arms in the transverse plane are defined to be 0° when the arms are directly out in front of the body. Positive angles are defined when the arms are angled to the sides of the body as shown in FIG. 11. Negative angles are defined when the arms are angled across the front the body.

For all arm angles the left and right arm angles are measured independently and relative to the body torso as opposed to gravity.

Regardless of the embodiment of the invention that is being used, a number of postures, positions and/or movements will have to be defined so that, given a set of angle and weight pressure data, meaningful data analysis can be performed. The following are example definitions:

Sitting:
Less than 10% of body weight on the feet
Right or left leg at 60° to 110°

Standing:
Right and left leg at ±10°
Full weight on the feet

Two-Point Kneeling, Position 1:
Less than 5% weight on the feet
Both legs at 0° to 15°

Two-Point Kneeling, Position 2:
Less than 5% weight on the feet
Both legs at 45° to 55°

One-Point Kneeling on Right Knee:
Right leg at ±10°
Left leg at 75° to 110°
5% to 75% of weight on left foot

One-Point Kneeling on Left Knee:
Left leg at ±10°
Right leg at 75° to 110°
5% to 75% of weight on right foot

Crouching:
Full weight on the feet
Both legs at 75° to 115°

Walking:
Alternate weight bearing between feet
At alternate times full weight is on each foot
Legs alternating at least ±10°

The angular position of the arms are defined to be relative to the person's torso position and therefore the position of the arms are defined as follows:

Sagittal Angle of Arms:
Sagittal angle of arms relative to gravity minus sagittal angle of upper back.

Frontal Angle of Left Arm:
Frontal angle of left arm relative to gravity minus frontal angle of upper back.

Frontal Angle of Right Arm:
Frontal angle of right arm relative to gravity plus frontal angle of upper back.

Transverse Angle of Arms:
Transverse angle of each arm relative to magnetic North minus transverse angle of upper back.

In addition to the above body positions, a “calibrate” position may be defined to be a person standing straight with legs directly underneath the body and arms hanging freely on the sides. This position can be used to establish what sensor readings correspond to zero angles.

Further definitions will be readily apparent to persons skilled in the art.

Portable Three-Dimensional Angle Sensor The following methodology assumes that each 3-D sensor 18 includes:

2 ±2 g dual-axis accelerometers (ADXL202E or similar);
(b) 3 ±300°/s single-axis rate gyroscopes (ADXRS300 or similar); and
(c) 2 dual-axis compass chips.

Preferably, all components are placed as close as possible to one another to reduce Ω²r acceleration. In addition, lines extending through the centers of the gyros 70 and accelerometers 60 perpendicular to their surfaces should intersect at a common point, as shown in FIG. 3.

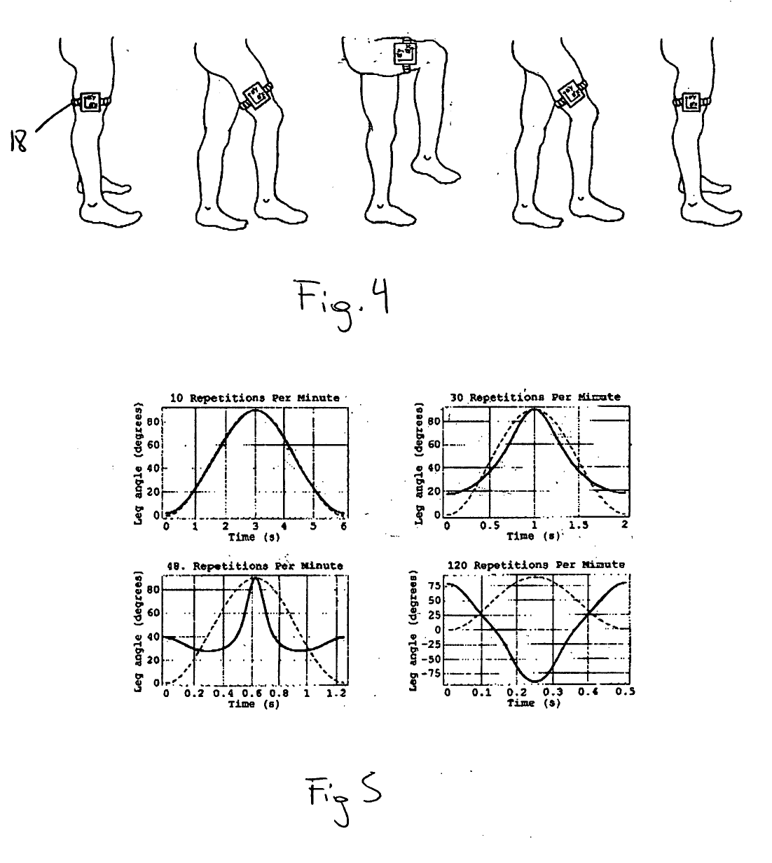

To perform a assessment of biomechanics, the angles between various joints on the human body must be accurately measured. It is common practice to measure angles relative to the direction of gravity using accelerometers. This method works particularly well in devices such as digital levels because the level is completely stationary when the angle measurement is taken. However, if a sensor is required to measure the angle of a patient's hip as he moves his leg up to 90 degrees and back down, (see FIG. 4) the sensor must compensate for the acceleration of the hip-mounted sensor. FIG. 5 shows the “angle” that would be sensed (solid line) by an accelerometer-based sensor if a patient's hip were moved from the vertical position of 0 degrees to a horizontal position of 90 degrees at the indicated repetition rate (10, 30, 48 and 120 repetitions per minute). It is assumed that the sensor is placed near the patient's knee, 40 cm from the point of rotation (see FIG. 4).

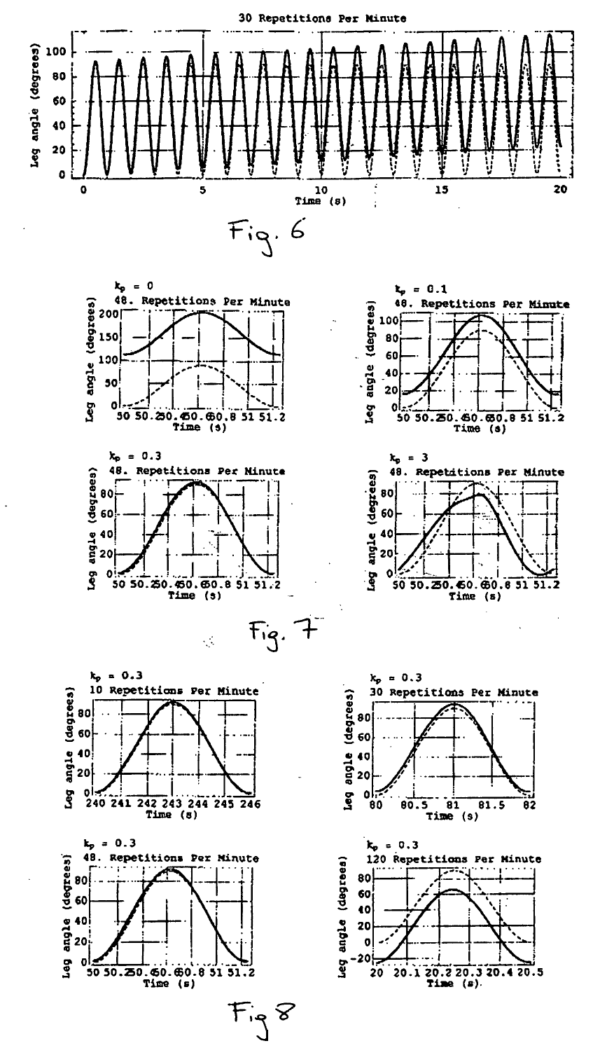

The difficulty with using a gyroscope to measure angles is that the gyroscope outputs a signal proportional to the rate of change of the angle. The signal must be integrated to get an angle measurement. The main problem that arises from this is that any zero-point offset produces an error that grows linearly in time. Eventually, one loses track of the absolute angle measurement. Referring to FIG. 6, wherein the solid line represents the “angle” sensed, the angle calculated by a gyroscope-based sensor can be simulated for a patient moving his leg between 0 degrees and 90 degrees at 30 repetitions per minute. For the sake of simplicity it is assumed that there is a 1% of Ωₘₐₓ (2.8°/s) offset and a 1% slope error (due to perhaps linear acceleration or inaccuracy in the device). The angle estimate obtained using the gyroscope-based sensor is very different from what was obtained using an accelerometer. The AC component is much more accurate except there is DC drift.

In the FAB sensor 18 of the present invention, both AC accuracy and DC stability are required. The following discussion describes how a gyroscope can be used in conjunction with an accelerometer to create a sensor 18 with good AC accuracy and no DC drift.

In the acceleration-tolerant angle sensor 18 of the present invention, a gyroscope is used for AC angle accuracy and an accelerometer is used to provide “feedback” that ensures DC stability. To estimate the angle, a method not unlike integral control is used. The estimate of the tilt angle, θ(t), is defined mathematically as follows:
θ(t)=∫Ω(t)dt+k ∫e(t)dt,
where
e(t)=θₐ(t)−θ(t),
and
θₐ(t)=arctan(dx/dy).

The estimate of the tilt angle, θ(t), is equal to the integral of the angular velocity signal from the gyroscope plus a term proportional to the integral of the “error”. Error in this sense is a misnomer: it is defined as the angle approximation from the accelerometers, θₐ(t), minus the estimate of the tilt angle θ(t). There is no guarantee that at any point in time θₐ(t) is a better approximation of the tilt angle than θ(t), it is simply that θₐ(t) is guaranteed to have no drift.

This angle estimation method can be applied to the case of a patient's leg moving between 0 and 90 degrees (see FIG. 4). FIG. 7 shows a plot of the estimated angle (solid line) versus the actual angle (dashed line) at various values of k. Except for the first graph, all graphs show the estimated angle (solid) versus the steady-state results. In the first graph, there is no steady-state because the error in the estimated angle drifts linearly with time.

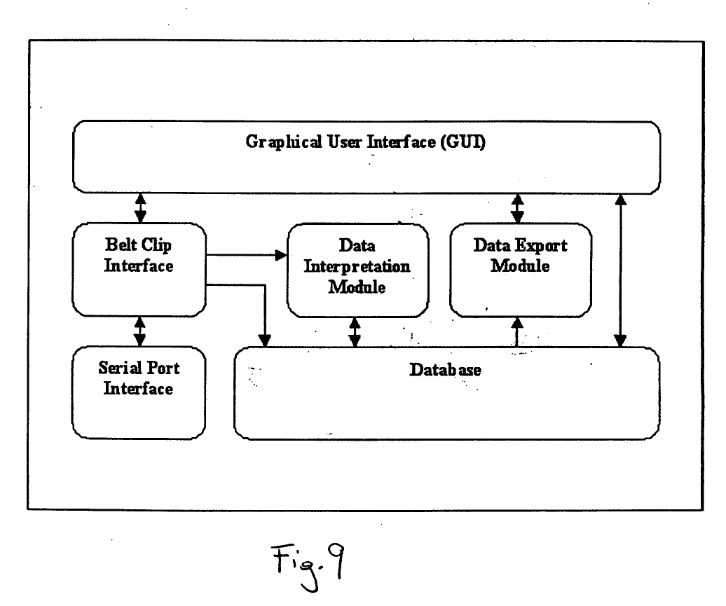

At low values of k, the steady-state error is inversely proportional to k and directly proportional to the offset in the gyroscope output. At high values of k, the sensor behaves like a pure accelerometer-based angle sensor. The optimum value for k therefore depends on how good the accelerometer estimate of the angle is and how small the drift in the integrated gyroscope angle estimate. A small offset in the gyroscope output and a poor accelerometer estimate would suit a small value for k. A large offset in the gyroscope output and an excellent accelerometer estimate would suit a large value for k. These effects are shown graphically in FIG. 8.

These results show how a gyroscope and accelerometer can be used together to measure tilt angles in a way that is neither sensitive to acceleration of the sensor nor sensitive to offset in the gyroscope signal. The sensor tracks the tilt angle with AC accuracy similar to a pure gyroscope-based angle sensor and the DC stability of a pure accelerometer-based tilt sensor.

Computation

The present invention comprises, in part, a high-bandwidth, acceleration-insensitive, three-dimensional (3D) orientation sensor 18. The invention utilizes sensors 18 having orthogonal magnetometers, accelerometers, and gyroscopes (see FIG. 3) and a novel computation to convert the readings into a 3×3 matrix that represents the orientation of the sensor relative to earth. The computation is based on using the gyroscopes to quickly track angular changes and then accelerometers and magnetometers to correct for any drift encountered through the gyroscope “integration” process.

As the computation makes extensive use of rotation matrices, a review of their characteristics is provided below in sections labeled I-V.

Section I provides a review of the characteristics of rotation matrices and Section II provides a description of how gyroscopes can be used to track changes in 3D angular position: each step of the computation the gyroscope angular velocity readings are used to rotate the orientation matrix by the amount implied by the readings. Although this technique produces excellent angular and temporal resolution, it suffers from drift.

Section III describes how accelerometers and magnetometers can be used to redundantly estimate the angular position of the sensor: after readings from these devices are used to estimate the gravity and magnetic field vectors, a Gram-Schmidt process is applied to construct a matrix that also estimates orientation. Although this method is highly sensitive to spurious accelerations, it does not suffer from drift.

Section IV discloses how data from gyroscopes, accelerometers, and magnetometers can be processed together to achieve high-bandwidth, acceleration-insensitive, drift-free angle measurements. The algorithm is based on tracking the orientation of the sensor with the gyroscopes but also “pulling” the orientation matrix slightly towards the accelerometer/magnetometer estimation each step of the computation.

Section V provides a description of some of the testing that has been completed and limitations of the device.

I. The Linear Algebra of Orientation Matrices

Three-dimensional rotation or “orientation” matrices are used extensively in the computation. The symbol R is used to denote all such matrices. To describe what an orientation matrix is, assume that there exist two frames: Frame 0 and Frame A. Frame 0 is the reference frame from which the orientation of Frame A is measured. The following matrix specifies the orientation of Frame A with respect to Frame 0:
^{Frame measured from→0}R_{A←Frame being measured} (1)

The superscript to the left denotes the frame in which the orientation is measured from; the subscript to the right denotes the frame being measured. R is a 3×3 matrix whose columns represent vectors in Frame 0 aligned along the coordinate axis of Frame A. For example, the first column represents a vector in Frame 0 aligned along the x-axis of Frame A. Correspondingly, the rows of this matrix represent vectors in Frame A aligned along the coordinate axes of Frame 0. Because of this property of the matrix, its transpose specifies the orientation of Frame 0 as measured from Frame A:
^{T}R₀=Transpose(⁰R_{T}) (2)
Now suppose that there exists a second Frame B and its orientation is known with respect to Frame 0. Its orientation with reference to Frame A is then
^{A}R_{B}=^{A}R₀ ⁰R_{B}. (3)
Note the cancellation of the adjacent 0's.

In the angle sensor calculation described in the following sections, Frame 0 is used to denote a reference frame fixed on earth, Frame A is used to denote the sensor's frame, and Frame B is used to denote the sensor's orientation as implied by the accelerometer/magnetometer readings. Certain orientation matrices are used extensively and are given names other than R: matrix A denotes the orientation of the sensor (Frame A) measured from earth, in other words
A=⁰R_{A}. (4)

Matrix B denotes the orientation as specified by the accelerometer/magnetometer readings (Frame B) measured from earth:
B=⁰R_{B}. (5)

Matrix S specifies the orientation of Frame B with respect to Frame A:
S=^{A}R_{B}=^{A}R₀ ⁰R_{B}=A^{T}B. (6)
Finally, the matrix

$${}_{}^{f}{R}{}_{f + Δ}^{} ( d Ω ) = ( \begin{matrix}1 & - d {Ω}_{z} & d {Ω}_{y} \\ d {Ω}_{z} & 1 & - d {Ω}_{x} \\ - d {Ω}_{y} & d {Ω}_{x} & 1\end{matrix} ) \qquad ( 7 )$$

denotes the orientation of a frame rotated infinitesimally from the (arbitrary) frame f about the vector dΩ by an angle (in radians) equal to the magnitude of the vector. The proof follows easily by considering infinitesimal rotations about each of the axes, and also observing that the sequence is unimportant for infinitesimal rotations.

It is clear that all 9 parameters in the orientation matrices are not independent degrees of freedom. By viewing the rotation matrix as 3 column vectors representing the rigid coordinate axes, then each of these vectors must have unit length and each of these vectors must be orthogonal to the others. These conditions impose 6 constraints on the 9 parameters resulting in 3 degrees of rotational freedom, as expected. Following from these properties, the matrices are orthogonal and normal: the dot product of any column with any other column, or any row with any other row is zero. The sum of the squares of any column or row as well as the determinant is unity.

A common method of specifying the orientation of a frame with 3 parameters is to use yaw-pitch-roll Euler angles. The yaw, pitch and roll angles can be thought of as instructions for how to rotate a frame initially coincident with the reference frame to its new orientation. This convention assumes first a counterclockwise rotation about the reference frame's z-axis by the yaw angle φ, followed by a counterclockwise rotation about the intermediate y-axis by the pitch angle θ, followed by a counterclockwise rotation about the new x-axis by the roll angle ψ. The Euler angles can be found by performing the following trigonometric operations on the elements of the rotation matrix:

$$ϕ = arctan ( {R}_{21} , {R}_{11} ) \\ θ = arctan ( - {R}_{31} , \sqrt{{R}_{21}^{2} + {R}_{11}^{2}} ) . \\ ψ = arctan ( {R}_{32} , {R}_{33} ) \qquad ( 8 )$$

The arctan(y, x) function is the four-quadrant inverse tangent and Rᵢⱼ is the element of R in the ith row and jth column.

II. Gyroscope Orientation Tracking

Rotation About a Fixed Axis

Gyroscopes provide accurate angular velocity information from which angular displacement can be determined. In the case of rotation about a fixed axis, the rotation angle θ as a function of time can be found directly with the integration

$$θ ( t ) - {θ}_{0} = {\int }_{0}^{t} ω ( t ) ⅆ t . \qquad ( 9 )$$

The integration is often performed discretely, in which case the relation for θ becomes
θᵢ₊₁=ΩᵢΔt+θᵢ. (10)
where Ωᵢ is the angular velocity averaged for at least twice the length of the sampling period to satisfy the Nyquist criteria.

Two limitations inherent in gyroscope-based angle sensors can be seen. First, an initial angle is required to start the procedure; if this angle is incorrect, then future angle measurements will likewise be incorrect. Second, during each step in the procedure a small amount of error is compounded to the angle estimation. Even if the errors are completely random, the angle measurement will undergo a random walk and deviate until it becomes meaningless.

3-D Rotation

When the axis of rotation is not fixed, the process of updating the sensor orientation becomes more involved. The novel method developed involves tracking the orientation matrix A (defined in Section I, above). The orientation of A is updated each step of the computation via multiplication with the infinitesimal rotation matrix
⁰A_{α+Δ}=⁰Aₐ ^{a}R_{a+Δ}(d/Ω) (11)

The vector dΩ=(dΩₓ dΩ_{y} Ω_{z})^{T} points along the instantaneous axis of rotation, has magnitude equal to the total angle of rotation, and is related to the average angular velocity during the time interval in question:

$$d {Ω}^{g} = ( \begin{matrix}d {Ω}_{x}^{g} \\ d {Ω}_{y}^{g} \\ d {Ω}_{z}^{g}\end{matrix} ) = Δ t ( \begin{matrix}{ω}_{{x}^{′}} \\ {ω}_{{y}^{′}} \\ {ω}_{{z}^{′}}\end{matrix} ) \qquad ( 12 )$$

The superscript g is used to indicate that this rotation vector is due to the gyroscopes (and will become important in Section IV, below). To carry out this calculation, a controller may poll the three gyroscopes to determine dΩ^{g}, multiply the A matrix by the infinitesimal rotation matrix implied by dΩ^{g}, and finally update A by setting it equal to the product.

III. Accelerometer/Magnetometer Orientation Estimation

Heading and Tilt Angle Measurements

For measuring heading, two orthogonal magnetometers with axes in a plane parallel to the horizontal are often used. For measuring tilt angle, two orthogonal accelerometers with axes in a plane perpendicular to the horizontal are typically employed. In both cases, an angle relative to magnetic north (heading) or vertical (tilt) can be determined by appropriately taking a four-quadrant arctangent of the two orthogonal sensor readings.

3-D Angle Measurements

For measuring the complete 3D angular orientation of a static body, three perpendicular accelerometers and three perpendicular magnetometers are required (see FIG. 3). For present purposes, the simplification will be made that earth's magnetic field points due north.

To describe the calculation, first define an orthogonal coordinate system (i.e. a reference frame) that is fixed with the earth such that the x-axis points east, the y-axis points north, and the z-axis points up. Next define an orthogonal coordinate system that is fixed with the sensor consisting of axes x′, y′, z′. Assume that a magnetometer as well as an accelerometer is aligned along each of the sensor's coordinate axes.

Each accelerometer reads the component of the reference frame's z-axis (i.e. up) aligned along its axis. Defining a_{x′}, a_{y′} and a_{z′} as the accelerometer readings along the respective sensor axes, the vector

$$a = ( \begin{matrix}{a}_{{x}^{′}} \\ {a}_{{y}^{′}} \\ {a}_{{z}^{′}}\end{matrix} ) \qquad ( 13 )$$

then approximates the direction of the earth's z-axis as measured in the sensor's frame.

Similarly, each magnetometer reads the component of the earth's magnetic field aligned along its axis. Defining b_{x′}, b_{y′} and b_{z′} as the magnetometer readings along the respective sensor axes, the vector

$$b = ( \begin{matrix}{b}_{{x}^{′}} \\ {b}_{{y}^{′}} \\ {b}_{{z}^{′}}\end{matrix} ) \qquad ( 14 )$$

then approximates the direction of earth's magnetic field as measured by the sensor.

As customary, define i, j, k as three unit vectors along the x, y, and z axes of the reference frame fixed on earth. Since a and b are not guaranteed orthogonal (because the magnetic field vector may point into the ground and the gravity vector may be affected by acceleration), i, j, k must be approximated using the Gram-Schmidt process:

$$i = \frac{b \times a}{\vert b \times a \vert} \\ j = \frac{a \times i}{\vert a \times i \vert} \\ k = i \times j . \qquad ( 15 )$$

It is crucial that the gravity vector is used in the second step of Eq. (15) to prevent problems associated with the inclination of earth's magnetic field. If a matrix is constructed using the unit vectors to form the columns, then the matrix will represent the orientation of the earth's reference frame as measured by the sensor, i.e. ^{B}R₀. It is more convenient to know the orientation of the sensor with respect to the reference frame, i.e. ⁰R_{B} or B. The unit vectors form the rows of this matrix (via the transpose property of orientation matrices)

$$B = {( \begin{matrix}i & j & k\end{matrix} )}^{T} = ( \begin{matrix}{i}_{{x}^{′}} & {i}_{{y}^{′}} & {i}_{{z}^{′}} \\ {j}_{{x}^{′}} & {j}_{{y}^{′}} & {j}_{{z}^{′}} \\ {k}_{{x}^{′}} & {k}_{{y}^{′}} & {k}_{{z}^{′}}\end{matrix} ) . \qquad ( 16 )$$

The primary problem with this type of accelerometer-based sensor is that it is sensitive to acceleration. In the non-accelerating case, the apparent acceleration is due only to gravity and the vector points straight up. However, in the accelerating case, this vector is completely arbitrary and the estimation of “up” is meaningless.

IV. Hybrid Solution

The pure gyroscope computation requires information that describes the initial 3D orientation of the sensor—it is not self-stabilizing. In fact it is unstable: the angular measurements drift without bound from even the smallest measurement errors. The magnetometer/accelerometer method has the advantage of stability. However, since the assumed direction for “up” is based on the accelerometer readings, it is strongly influenced by any acceleration experienced by the sensor.

A method that combines the high-bandwidth, acceleration-insensitive angle measurements of the gyroscope technique with the stability of the accelerometer/magnetometer technique is required if the device is to be used to track human body orientation during regular activities.

Computation Overview

In the hybrid solution the vector used to rotate the orientation matrix is the sum of the rotation vector from the gyroscopes and a vector that “pulls” slightly towards the accelerometer/magnetometer orientation estimation. The hybrid solution confers excellent sensitivity to small or rapid changes in orientation and eliminates drift.

Computation Details

The computation of the hybrid solution executes at a constant rate with frames separated by the time period Δt. A 3×3 matrix denoted A (initially set to the identity matrix) is used to track the orientation of the sensor. The stability of the computation ensures that the A matrix will converge to represent the correct sensor orientation.

Every sampling period, the angular velocity vector, Ω, is measured using the onboard rate gyroscopes to calculate the gyroscope rotation vector via Eqn. (11). To correct for integration drift, A is rotated slightly towards the feedback matrix B found from the compass and accelerometer readings via Eqns. (13)-(16). Before the A matrix can be rotated towards B, a vector specifying the desired correction must be determined. The magnitude of this rotation (the length of the desired vector) is proportional to the total angle, Φ, separating A and B.

Since S specifies the orientation of Frame B as measured from Frame A, it must contain information about the total angle of rotation between the frames as well as the axis of rotation. It is always possible via a suitable similarity transformation to change to a coordinate system where the rotation S′ is entirely about the new z-axis:

$${S}^{′} = ( \begin{matrix}cos Φ & sin Φ & 0 \\ - sin Φ & cos Φ & 0 \\ 0 & 0 & 1\end{matrix} )$$

The trace of S′ is
TrS′=2 cosΦ+1
but since the trace of a matrix is invariant under similarity transformation
TrS=2 cosΦ+1
solving for the total angle gives

$$Φ = arccos ( \frac{TrS - 1}{2} ) \qquad ( 17 )$$

where Tr( ) is the trace or the sum of the diagonal elements of the matrix and S specifies the orientation of Frame B as measured from Frame A as per Eqn. (6).

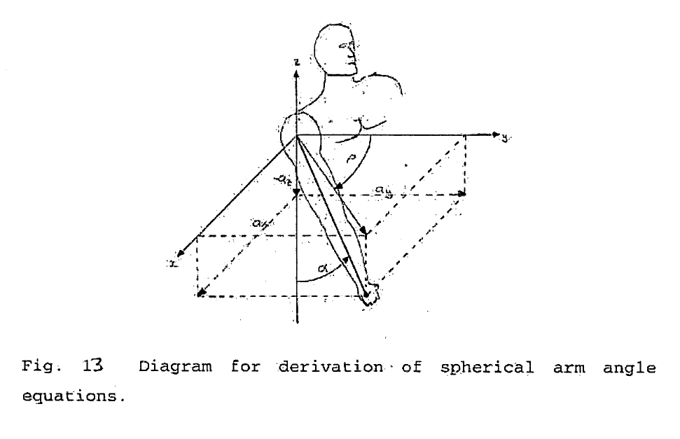

Consider the rotation of a vector r about n by the finite angle Φ. Referring to FIG. 12, the rotated vector r′ can be described by the equation
r′=n(n·r)+[r−n(n·r)]cosΦ+(r×n)sinΦ
which after a slight rearrangement of the terms leads to
r′=r cosΦ+n(n·r)[1−cosΦ]+(r×n)sinΦ. (18)
The formula can be cast into a more useful form by introducing a scalar e₀ and a vector e with components e₁, e₂, and e₃ defined as

$$\begin{matrix}{e}_{0} = \frac{cos Φ}{2} \\ and & ( 19 ) \\ e = \frac{n sin Φ}{2} . & ( 20 )\end{matrix}$$

Since |n|=1, these four quantities are obviously related by
e₀²+|e|²=e₀²+e₁²+e₂²+e₃²=1
It follows that

$$cos Φ = 2 {e}_{0}^{2} - 1.$$

$$and$$

$$n = \frac{2 {e}_{0}}{sin Φ} e .$$

With these results, (18) can be rewritten as
r′=r(e₀²−e₁²−e₂²−e₃²)+2e(e·r)+2(r×e)e₀ (21)
Equation (21) thus gives r′ in terms or r and can be expressed as a matrix equation
r′=S·r
where the components of S follow from inspection

$$S = ( \begin{matrix}{e}_{0}^{2} + {e}_{1}^{2} - {e}_{2}^{2} - {e}_{3}^{2} & 2 ( {e}_{1} {e}_{2} + {e}_{0} {e}_{3} ) & 2 ( {e}_{1} {e}_{3} - {e}_{0} {e}_{2} ) \\ 2 ( {e}_{1} {e}_{2} - {e}_{0} {e}_{3} ) & {e}_{0}^{2} - {e}_{1}^{2} + {e}_{2}^{2} - {e}_{3}^{2} & 2 ( {e}_{2} {e}_{3} + {e}_{0} {e}_{1} ) \\ 2 ( {e}_{1} {e}_{3} + {e}_{0} {e}_{2} ) & 2 ( {e}_{2} {e}_{3} + {e}_{0} {e}_{1} ) & {e}_{0}^{2} - {e}_{1}^{2} - {e}_{2}^{2} + {e}_{3}^{2}\end{matrix} ) .$$

Equations (17) and (19) can be used to solve explicitly for e₀, resulting in

$${e}_{0} = \frac{\sqrt{TrS + 1}}{2} \qquad ( 22 )$$

Knowing e₀, the components of e can now be found by examining the elements of S. For instance, e₁ can be found noting that
S₂₃−S₃₂=2(e₂e₃+e₀e₁)−2(e₂e₃−e₀e₁)
and then solving for e₀

$${e}_{1} = \frac{{s}_{23} - {s}_{32}}{4 {e}_{0}}$$

After solving for e₂ and e₃ in the same manner, the vector e can be constructed

$$e = \frac{1}{4 {e}_{0}} ( \begin{matrix}{S}_{23} - {S}_{32} \\ {S}_{31} - {S}_{13} \\ {S}_{12} - {S}_{21}\end{matrix} ) \qquad ( 23 )$$

To find n rather than e, (17), (20) and (22) can be used to eliminate the total angle and eₒ. The desired results emerges:

$$n = \frac{1}{\sqrt{( 1 + TrS ) ( 3 - TrS )}} ( \begin{matrix}{S}_{23} - {S}_{32} \\ {S}_{31} - {S}_{13} \\ {S}_{12} - {S}_{21}\end{matrix} ) \qquad ( 24 )$$

where Sᵢⱼ is the element of S in the ith row and jth column. The desired vector specifying the small rotation is thus
dΩ^{c}=kΔtΦn (25)
where k is a gain parameter used to tune the feedback for optimum performance. A larger value of k pulls the orientation matrix towards the accelerometer/magnetometer approximation quickly. For stability kΔt<1. The superscript c is used to indicate that this rotation vector is the correction term.

Equation (25) can be written more explicitly using Eqns. (16) and (24) as

$$d {Ω}^{c} = \frac{k Δ t arccos ( \frac{TrS - 1}{2} )}{\sqrt{( 1 + TrS ) ( 3 - TrS )}} ( \begin{matrix}{S}_{23} - {S}_{32} \\ {S}_{31} - {S}_{13} \\ {S}_{12} - {S}_{21}\end{matrix} ) \qquad ( 26 )$$

Since both rotation vectors dΩ^{g} and dΩ^{c} are small, they can be added to get the vector specifying the total rotation:
dΩ=dΩ^{g}+dΩ^{c}. (27)
Since dΩ is also small, the infinitesimal matrix rotator, (see Eqn. (7)) can be used to execute the rotation. The new orientation of the sensor is therefore given by Eqn. (11)
⁰A_{A+Δ}=⁰A_{A} ^{A}R_{A+Δ}(dΩ). (28)
remembering that dΩ included both the gyroscope rotation and a rotation towards the accelerometer/magnetometer feedback matrix. This computation executes at a rapid rate to accurately track the angular orientation of the sensor.

Since the rotation is not truly infinitesimal, it is necessary to orthonormalize A occasionally via a Gram-Schmidt process such as Eqn. (15). As the calculation executes, A converges to the correct orientation at the time constant k⁻¹. After convergence, the columns of A represent the sensors coordinate frame axes as measured in the reference frame on earth.

Implementation Details

The choice of k depends on both how quickly the angle measurements drift with zero feedback and how much acceleration the sensor experiences. In a case where the angle measurements drift slowly and the sensor experiences a great deal of spurious acceleration, a very small time constant is suitable and the sensor will behave more like a pure gyroscope angle sensor. In the other extreme, where the gyroscope angle measurements drift very fast and the system is subject to negligible accelerations, a large value of k is preferred and the sensor behaves similar to a pure accelerometer/magnetometer angle sensor. Testing of a prototype angle sensor gave best results with a time constant of 2 seconds (k=0.5).

To expedite the convergence of the A matrix, it was found convenient to increase k at start up and allow the computation to proceed with a large value for a couple of time constants. Another implementation note is that numerical precision effects will sometimes cause the trace of the S matrix to lie outside the range of −1 to +3 and thus cause the total angle of rotation to be imaginary. Forcing the trace of S to be within its expected range eliminated this problem. Finally, when calculating S one has the choice of using the current B matrix or the previous B matrix. This choice was found to be unimportant and the most current matrix was used out of convenience.

A prototype sensor was built and used to test the computation. The sensor readings were routed into a computer, which then performed the calculation in real time. The program converted the orientation matrix to yaw, pitch and roll angles and displayed them in real time on the screen. The sensor was held in a fixed position and shaken to check the devices sensitivity to acceleration. No noticeable effect from the acceleration was present.

Relative Angle Calculation for the FAB System

This section presents the calculations used to extract the body angles of a human subject wearing the FAB system from the orientation matrices stored in each of the 3-D angle sensors. The angles are reported “relative” to the subject in a convenient way as to ease interpretation of the data. Three-dimensional angle sensors, such as those described above, can be mounted to a human patient and used to track his motion. Because the human arms and legs contain only revolute joints, their position is specified by the angles of the limb segments or the joint coordinates. 3-D angle sensors provide an orientation relative to a fixed reference frame on earth. However, to transform these absolute angle measurements into meaningful joint coordinates, it is more useful to measure angles relative to the human subject.

This section describes the relative angle calculation performed in the Function Assessment of Biomechanics (FAB) system. In the preferred embodiment the FAB system employs six 3-D angle sensors attached to the patient's lower back, spine, right and left upper arms, and right and left thighs. Rather than reporting angles relative to the earth, the FAB system processes the raw orientation data from the sensors to calculate relative angles between body parts. This provides information that specifies the position of the person and their limbs in an easier-to-understand format.

Section V describes the calculation used to determine the relative orientation between two 3-D angle sensors. Section VI then shows how the angles for all of the sensors are calculated from these relative orientation matrices.

V. Relative Orientation Calculation

In the preferred embodiment, the FAB system monitors the orientation of the patient's lower back, spine, right upper arm, left upper arm, right thigh and left thigh using six 3-D angle sensors. For the purposes of this section, “patient position” refers to a point in 14-dimensional hyperspace where each coordinate represents one of the measured angles. Through the development of the present invention, a means for presenting patient position in an easily-understandable way was established. The concept revolves around providing body angles relative to other parts of the body. For example, the angles of the arm are measured relative to the sensor mounted to the patient's spine so that the arm angles read the same value when the shoulder joint is in the same position, regardless of whether the patient is standing straight or leaning forward.

It was shown above that each angle sensor contains a 3×3 matrix that specifies its orientation relative to the earth. To calculate the desired “relative” body angles, a method must first be established for constructing a relative orientation matrix for each sensor that specifies its orientation relative to a second “reference” angle sensor. The desired body angles can then be extracted from these matrices using the four-quadrant arctangent function.

### An Illustrative Example: Right Arm Orientation

As already mentioned, the orientation of the right arm is measured relative to the spine. Let Frame 0 be the earth frame, Frame SP be the spine sensor frame, and Frame RA be the right arm sensor frame. Thus ⁰R_{SP} and ⁰R_{RA} represent the orientation of the spine and right arm frames relative to earth, respectively. These are the “orientation” matrices stored automatically by the sensors according to the calculations in IV above. Naively, it would seem that the desired “relative” orientation matrix is the matrix that specifies the orientation of Frame RA relative to Frame SP. However, this is not the case. It is not known how the arm sensor will be attached to the patient. The arm sensor must be “calibrated” so that the relative orientation matrix is equal to the identity matrix when the patient is in the neutral position (standing tall, arms by his side).

An additional frame that represents the calibrated arm sensor, Frame RA-C, must be defined (the “-C” indicates a calibrated frame). At calibration time, Frame RA-C is defined to be coincident with Frame SP of the spine. What happens, mathematically, is that this calibrated frame is “glued” rigidly to Frame RA such that the two frames move together. Body angles are then extracted from the orientation of Frame RA-C so that the angles read zero at the neutral position.

Since Frame RA-C is “glued” to Frame RA, it always has the same orientation relative to Frame RA. The matrix ^{RA}R_{RA-C} describes this relationship and can be thought of as the calibration matrix. At calibration time, Frame RA-C is defined to be coincident with Frame SP. From this fact, the calibration matrix can be calculated
^{RA}R_{RA-C}=^{RA}R_{SP}=^{RA}R₀ ⁰R_{SP} (at calibration) (29)
We know that ^{RA}R₀ is the transpose of the A matrix for the right arm sensor and ⁰R_{SP} is the A matrix for the spine sensor (see sections I-IV above). We can write:

$${C}_{RA} = {{A}_{RA}}^{\%} {A}_{SP} ( at calibration ) \qquad ( 30 )$$

The over-tilde is used to represent the transpose of a matrix. Now at any subsequent time, the desired relative angles will be contained in the matrix ^{SP}R_{RA-C} found via
^{SP}R_{RA-C}=^{SP}R₀ ⁰R_{RA}^{RA}R_{RA-C} (31)
which, using the notation from sections I-IV, is equivalent to

$${R}_{RA} = {{A}_{SP}}^{\%} {A}_{RA} {C}_{RA} \qquad ( 32 )$$

The underscript R is new notation: it represents the relative orientation matrix that is actually used to extract the body angles.

The calculation of the relative matrix for the other sensors follows by replacing RA with the sensor in question and SP with its reference sensor.

TABLE 1
Relative Angle Summary

| | Referenced | | | | Neutral |
|---|---|---|---|---|---|
| Sensor | to | Angle | Equation | Range | position |
| Lower | Earth | Yaw/ | φ = arctan(R₁₂, R₂₂) | {0°, | Arms by |
| back | | heading | | 360°} | patient's |
| | | Pitch | θ = arctan(R₃₂, \sqrt{R₁₂² + R₂₂²}) | {−180°, | side, |
| | | | | 180°} | back |
| | | Roll | ψ = arctan(−R₃₁, R₃₃) | {−180°, | straight, |
| | | | | 180°} | legs |
| Spine | Lower back | Yaw | φ = arctan(R₁₂, R₂₂) | {−180°, | straight |
| | | | | 180°} | and kness |
| | | Pitch | θ = arctan(R₃₂, \sqrt{R₁₂² + R₂₂²}) | {−180°, | locked. |
| | | | | 180°} | |
| | | Roll | ψ = arctan(−R₃₁, R₃₃) | {−180°, | |
| | | | | 180°} | |
| Right | Spine | Polar | ρ = arctan(−R₁₃, −R₂₃) | {−90°, | |
| arm | | | | 270°} | |
| | | Azimuth | α = arctan(\sqrt{R₁₃² + R₂₃²}, R₃₃) | {0°, | |
| | | | | 180°} | |
| Left | Spine | Polar | ρ = arctan(R₁₃, −R₂₃) | {−90°, | |
| arm | | | | 270°} | |
| | | Azimuth | α = arctan(\sqrt{R₁₃² + R₂₃²}, R₃₃) | {0°, | |
| | | | | 180°} | |
| Right | Lower back | Polar | ρ = arctan(−R₁₃, −R₂₃) | {−90°, | |
| leg | | | | 270°} | |
| | | Azimuth | α = arctan(\sqrt{R₁₃² + R₂₃²}, R₃₃) | {0°, | |
| | | | | 180°} | |
| Left | Lower back | Polar | ρ = arctan(R₁₃, −R₂₃) | {−90°, | |
| leg | | | | 270°} | |
| | | Azimuth | α = arctan(\sqrt{R₁₃² + R₂₃²}, R₃₃) | {0°, | |
| | | | | 180°} | |

VI. Body Angle Calculations

Using the procedure from Section V, a relative orientation matrix (an underscript R) for each sensor can be constructed. Calibrated body angles can then be extracted from this matrix. Table I shows the frame that each of the FAB sensors is referenced to and summarizes the angle equations that will be derived next.

A. Lower Back Sensor

The lower back sensor is used to track the absolute orientation of the subject. Since it is referenced to the earth, no calibration is required and the A matrix contained in the sensor is used “as is” to extract the angles (the A matrix is the desired underscript R matrix). Three Euler angles are used to specify the orientation of the lower back sensor. The angles represent “instructions” for how the subject could have moved to get into his current position. The sequence of these rotations is important. The first rotation is a change in heading or a yaw. The yaw angle specifies the direction that the patient is facing: 0 degrees is magnetic north and the angle grows as the patient turns to face east, south, west and reaches 359 degrees as he approaches north again (i.e. the positive direction represents twists to the right). The second angle is the pitch and describes whether the patient is leaning forward or backward; positive angles indicate a backward lean such that the subject's chest is facing up towards the sky. The final angle is the roll and describes whether the patient is leaning to the right or to the left; positive angles indicate leans to the right and negative angles indicate leans to the left.

How are the desired angles extracted from the relative orientation matrix? To find out, define four frames: the reference frame on earth, Frame 0; the sensor's frame after the heading change, Frame 1; the sensor's frame after the heading change and pitch, Frame 2; and finally the sensor's frame after the heading change, pitch and roll, Frame 3.

The orientation of Frame 1 relative to Frame 0 can be found by noting that the heading is positive when the patient rotates clockwise (watching from above). Frame 1's x-axis picks up a negative y component (cosφ −sinφ 0)^{T}, Frame 1's y-axis picks up a positive x component (sinφ cosφ 0)^{T}, and Frame 1's z-axis is unchanged. Remembering that the columns of the orientation matrix represent the coordinate axes of the rotated frame, the matrix that specifies the orientation of Frame 1 with respect to Frame 0 is thus

$${}_{}^{0}{R}{}_{1}^{} = ( \begin{matrix}cos ϕ & sin ϕ & 0 \\ - sin ϕ & cos ϕ & 0 \\ 0 & 0 & 1\end{matrix} ) . \qquad ( 33 )$$

The next rotation is about the x-axis of Frame 1 by the pitch angle. Since a lean backwards towards the sky is defined as positive, as the sensor undergoes a small positive pitch its new x-axis is unchanged, its new y-axis picks up a positive z-component, and its new z-axis picks up a negative y-component. Frame 2 relative to Frame 1 is thus given by

$${}_{}^{1}{R}{}_{2}^{} = ( \begin{matrix}1 & 0 & 0 \\ 0 & cos θ & - sin θ \\ 0 & sin θ & cos θ\end{matrix} ) \qquad ( 34 )$$

The final rotation is about the y-axis of Frame 2 by the roll angle. Since a lean to the right is defined as positive, as the sensor undergoes a small positive roll, its new x-axis picks up a negative z-component, its new y-axis is unchanged, and its new z-axis picks up a positive x-component. Frame 3 relative to Frame 2 is thus given by

$${}_{}^{2}{R}{}_{3}^{} = ( \begin{matrix}cos ψ & 0 & sin ψ \\ 0 & 1 & 0 \\ - sin ψ & 0 & cos ψ\end{matrix} ) \qquad ( 35 )$$

The matrix that specifies the orientation of the lower back sensor relative to earth is therefore

$${R}_{LB} = {}_{}^{0}{R}{}_{3}^{} = {}_{}^{0}{R}{}_{1}^{} {}_{}^{1}{R}{}_{2}^{} {}_{}^{2}{R}{}_{3}^{} , \qquad ( 36 )$$

which after performing the matrix multiplication yields

$${R}_{LB} = ( \begin{matrix}sin ϕ sin θ sin ψ + cos θ cos ψ & sin ϕ cos θ & - sin ϕ sin θ cos ψ + cos θ sin ψ \\ - sin ϕ cos ψ + & cos ϕ cos θ & - cos ϕ sin θ cos ψ - \\ cos ϕ sin θ sin ψ & & sin θ sin ψ \\ - cos θ sin ψ & sin θ & cos θ cos ψ\end{matrix} ) \qquad ( 37 )$$

Noting that R₁₂/R₂₂=tanφ, the heading angle (0 to 360 degrees) is given by
φ=arctan(R₁₂,R₂₂). (38)
Similarly, the pitch and roll angles are given by
θ=arctan(R₃₂, \sqrt{R₁₂²+R₂₂²})
ψ=arctan(−R₃₁,R₃₃) (39)
Applying these formulae on the numerical elements of the lower back sensor's orientation matrix yield the desired angles.
B. Spine Sensor

The spine angles are defined exactly the same as the lower back angles; however, the spine angles are measured relative to the lower back sensor's coordinate frame rather than the earth's.

C. Right Arm Sensor

The arm angles are measured using standard spherical angles. The polar angle, ρ, measures the “heading” of the arm relative to the spine sensor. The azimuth angle, α, measures the angles between the arm and the vertical. Both angles are shown graphically on the patient in FIG. 13. To derive the arm angle equations, first define an “arm vector” parallel to the patient's arm with x, y and z components measured in the spine sensor's frame. Taking the appropriate arctangents of these components provides the desired angles. For the right arm, the polar angle is given by
ρ=arctan(αₓ, α_{y}). (40)
Now note that the “arm vector” is equivalent to the negative z-axis in the arm sensor frame. Since the third column of the relative orientation matrix specifies the components of the arms sensor's z-axis, the frontal angle can now be written in terms of the relative orientation matrix
ρ=arctan(−R₁₃,−R₂₃) (41)
In a similar fashion, the azimuth angle is given by
α=arctan(\sqrt{R₁₃²+R₂₃²}, R₃₃). (42)
Finally, to ease the interpretation of data, the software is designed to process the angle information and describe the planes (sagittal, frontal, and transverse) that the motion is occurring in.
D. Left Arm Sensor

The left arm is treated as a mirror image of the right arm and thus the x-matrix elements (elements with an index of 1) are negated. This simply results in a negative sign on the left arm polar angle.

E. Legs

The right and left leg angles are measured exactly the same as the right and left arm angles. The leg angles are referenced to the lower back sensor, however.

This section described the calculations used to extract patient body angles from the six 3-D angle sensors used by the FAB system of the present invention. An improvement could potentially be made by also calibrating the lower back and spine sensors relative to gravity, thus removing the need to mount these sensors in a specific orientation.

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments as fall within the true scope of the invention.

1. A system for determining a patient's body position, comprising:
a) at least two portable acceleration insensitive 3-dimensional sensors, said sensors fastenable to desired portions of said patient's body, wherein each of said 3-dimensional sensors has:
i. an accelerometer component operative to perform acceleration measurements along 3 orthogonal axes;
ii. a gyroscopic component operative to measure rotational velocity along said 3 orthogonal axes; and
iii. a magnetometer component operative to perform magnetic measurements along said 3 orthogonal axes;
b) a portable data memory unit, fastenable to said patient's body and connected to said sensors, said data memory unit for receiving and storing data from said sensors;
c) a data processing unit, for processing said data to determine relative positions of said sensors.

2. The system of claim 1, wherein each one of said 3-dimensional sensors has 2 accelerometers, 3 gyroscopes and 2 magnetometers, and wherein each one of said accelerometers and said magnetometer is capable of performing measurements along two of said axes.

3. The system of claim 1, wherein said data memory unit is connected to said sensors by a wireless connection.

4. The system of claim 1, wherein said data processing unit is a PC.

5. The system of claim 1, wherein said sensors are connected to said data memory unit in one of a daisy chain and a star configuration.

6. The system of claim 1, wherein said data memory unit is clipped or fastened to a belt of the patient.

7. The system of claim 1, wherein said data memory unit is capable of relaying said data to said data processing unit in real time.

8. The system of claim 1, wherein said system comprises 6 of said 3-D sensors, such that one of said 3-D sensors may be fastened to each of the patient's upper arms, upper legs, upper spine and lower spine.

9. The system of claim 1, wherein said system further comprises a foot sensor for placement under each of the patient's feet, wherein said foot sensors are connected to said data memory unit and are operative to sense pressure.

10. The system of claim 1, wherein said data memory unit stores said data from said sensors for delayed transference to said data processing unit.

11. The system of claim 10, wherein said data is transmitted to said data processing unit from said data memory unit one of wirelessly, through a cable, and with a memory card.

12. A system for determining a patient's body position, comprising:
a) at least two portable acceleration insensitive 3-dimensional sensors, said sensors fastenable to desired portions of said patient's body, wherein each of said 3-dimensional sensors has:
i. an accelerometer component operative to perform acceleration measurements along 3 orthogonal axes;
ii. a gyroscopic component operative to measure rotational velocity along said 3 orthogonal axes; and
iii. a magnetometer component operative to perform magnetic measurements along said 3 orthogonal axes;
b) a portable data memory unit, fastenable to said patient's body and connected to said sensors, for receiving and storing data from said sensors.

13. The system of claim 12, wherein each one of said 3-dimensional sensors has 2 accelerometers, 3 gyroscopes and 2 magnetometers, and wherein each one of said accelerometers and said magnetometer is capable of performing measurements along two of said axes.

14. The system of claim 12, wherein said data memory unit is connected to said sensors by a wireless connection.

15. The system of claim 12, wherein said data from said sensors is relayed to a data processing unit, either after a delay or in real time, wherein said data processing unit processes said data to determine relative positions of said sensors.

16. The system of claim 15, wherein said data processing unit is a PC.

17. The system of claim 12, wherein said sensors are connected to said data memory unit in one of a daisy chain and a star configuration.

18. The system of claim 12, wherein said data memory unit is clipped or fastened to a belt of the patient.

19. The system of claim 12, wherein said system comprises 6 of said 3-D sensors, such that one of said 3-D sensors may be fastened to each of the patient's upper arms, upper legs, upper spine and lower spine.

20. The system of claim 12, wherein said system further comprises a foot sensor for placement under each of the patient's feet, wherein said foot sensors are connected to said data memory unit and are operative to sense pressure.

21. A system for determining a patient's body position, comprising:
a) 6 portable acceleration insensitive 3-dimensional sensors, said 3-dimensional sensors fastenable to the patient's upper and lower spine, upper arms and upper legs, wherein each of said 3-dimensional sensors has:
i. an accelerometer component operative to perform acceleration measurements along 3 orthogonal axes;
ii. a gyroscopic component operative to measure rotational velocity along said 3 orthogonal axes; and
iii. a magnetometer component operative to perform magnetic measurements along said 3 orthogonal axes;
b) a portable data memory unit, fastenable to said patient's body and connected to said sensors, for receiving and storing data from said sensors.

22. The system of claim 21, wherein each one of said 3-dimensional sensors has 2 accelerometers, 3 gyroscopes and 2 magnetometers, and wherein each one of said accelerometers and said magnetometer is capable of performing measurements along two of said axes.

23. The system of claim 21, wherein said data memory unit is connected to said sensors by a wireless connection.

24. The system of claim 21, wherein said data from said sensors is relayed to a data processing unit, either after a delay or in real time, wherein said data processing unit processes said data to determine relative positions of said sensors.

25. The system of claim 24, wherein said data processing unit is a PC.

26. The system of claim 21, wherein said sensors are connected to said data memory unit in one of a daisy chain and a star configuration.

27. The system of claim 21, wherein said data memory unit is clipped or fastened to a belt of the patient.

28. The system of claim 21, wherein said system further comprises a foot sensor for placement under each of the patient's feet, wherein said foot sensors are connected to said data memory unit and are operative to sense pressure.
